(19) United States
(12) Patent Application Publication
(10) Pub. No.: US 2004/0225199 A1
(43) Pub. Date: Nov. 11, 2004

(54) Advanced physiological monitoring systems and methods

(76) Inventors: Shane Walter Evanyk, Plano, TX (US); Walter Reese Evanyk, Plano, TX (US); Mark Vernon Wilson, Dallas, TX (US)

Correspondence Address: Walter R. Evanyk, 3200 Sherrye Dr, Plano, TX, 75074-4693, US

(21) Appl. No.: 10/431,865

(22) Filed: May 8, 2003

(51) Int. Cl. A61B 5/00

(52) U.S. Cl. 600/300; 128/903

(57) ABSTRACT

A system for wirelessly monitoring, in real time, certain physiological/biological parameters of an animate body, such as an athlete or a patient. The parameters are sensed and measured by a transducer located on the animate body. The transducer is part of a transponder that includes an electronic unit containing a wireless receiver/transmitter. Remote R/T access units are spaced such that at any given time, signals from the transponder will be received by at least one of the access units where the data can be coupled to a display monitor and the measured parameters viewed in real time. In addition, a real time video image of an animate body, such as a patient/athlete, may be transmitted to a remotely located monitor for monitoring along with the transmitted physiological/biological patient parameters.

### BACKGROUND OF THE INVENTION

1. Field of the Invention

The present invention relates in general to physiological monitoring systems and methods and, in particular, to a transponder including an electronic circuit or unit, such as an RF unit, an IR unit, a Sonic unit, and the like, that is detachably coupled to a transducer affixed to an animate body (i.e. a person or animal) for measuring at least one physiological or biological parameter, the transducer wirelessly transmitting the measured parameter to a remote receiver station either at some timed interval or when interrogated (there being no direct physical connections existing between the transponder and the remote receiver station).

The invention also perceives the use of a video system selectively activated to transmit, wirelessly or by a hard-wired system, a real time view of the patient whose parameters are being monitored.

2. Background of the Invention

Systems that monitor certain physiological or biological parameters of a physical body are well known in the art. Treadmills and electrocardiograms and other systems for measuring and monitoring sleep abnormalities, oxygen use, blood pressure, temperature, and the like, are all known and used in the prior art. However, to the knowledge of the inventors, such systems are always physically connected by wires, cords, and the like to the person being monitored. During surgery, for example, such equipment for measuring physiological parameters is connected by cables between the patient and the monitoring device and these cables interfere with the movement and actions of the physicians, surgeons, and nurses in the operating room. In hospitals, patients in their rooms have their physiological parameters measured and monitored by systems that connect wires or cables to the patient and can carry the monitored parameters to a nurse's station or elsewhere. If the nurse, for any reason, has to move away from her station, important physiological information that comes from the patient to the station may be missed. Such information may be critical to the health status of the patient. For instance, heart or brain monitored signals may provide indication of the onset of a stroke or a heart attack. If the condition is immediately reviewed, or reviewed in real time, the life of the patient may be saved.

When athletes are performing their particular feats, it is only after the feat has been performed that physiological measurements can be taken to monitor the athlete's health condition. For instance, a runner, a football player, a basketball player, or other athlete may be having health problems that are not apparent until after the game or contest is over and the athletes vital statistics can be measured with the wires and cables connected to his body. In some cases, the athlete has died during or after the performance.

If the vital statistics had been available during the contest, perhaps the athlete could have been saved. In addition, such measurements taken during the actual performance of the individual can provide critical, real-time feedback information to a trainer, coach, physician, or the like. Obviously, such system could be used to monitor individuals under medical care or in rehabilitation.

Thus, it would be advantageous to have a first response system utilizing a wireless system for monitoring such physiological parameters so that bulky cables and wiring do not limit the usefulness of the parameter measuring system.

It would also be advantageous to have a real time video of the patient selectively transmitted, either by hard-wire or wirelessly, to a remote station for visual monitoring of the patient in real time.

### SUMMARY OF THE INVENTION

The present invention overcomes the disadvantages of the prior art by providing a wireless system for measuring and monitoring physiological and/or biological parameters of a physical body in real time. For purposes of simplicity, as used herein, the term “physiological” parameter is intended to include the terms “biological” parameter and “vital statistics”. All of these terms are intended to be interchangeable. For purpose of simplicity, the term “body” used herein id defined as “an animate body” including human, animal, and the like.

Thus, during the performance of the individual, the vital signs are being collected in real time and transmitted to a remote location for observation without the use of physical cables or wires directly connected between an individual and a monitor.

In like manner, during performance of surgery, the vital signs of the patient are measured during the surgery and then transmitted wirelessly to a monitor in the room or elsewhere where the vital signs are under constant review, in real time, by qualified personnel. A permanent record can be established for review at a later time.

Also, the vital signs of a patient in a hospital room may be measured in real time and transmitted wirelessly to the nurse's station, to a doctor's office, or to a portable monitor, such as a PDA, that can be carried by the nurse and/or doctor, and, the vital signs of the patient can be monitored, in real time, wherever the nurse or doctor is located. The vital signs can also be stored at a remote site.

In addition, the system may have a Local Positioning System (LPS) that identifies exactly where the individual, such as an athlete, patient, doctor, nurse, or any other desired individual who may be wearing a transponder, is physically located. In the case of a hospital patient, the nurse or doctor can proceed immediately to the patient room as necessary as in the case of an emergency. Further, when the doctor, nurse, or other qualified personnel, receives the data from the patient on a portable device, such as a PDA, as set forth above, the portable device may also transmit command signals to the patient's transponder, a nurse's station, a doctor's office, or the like to cope immediately with the emergency at hand.

Also, when the transponder is associated with a person such as a doctor or nurse within the confines of a structure such as a hospital, nursing home, rehabilitation center, and the like, the sensor is not needed. However, a unique code is assigned to and identifies that particular transponder so that the location of the person is always known.

Also, with this first response system, a real time video image of the patient may be selectively transmitted to a remote location either by wire or wirelessly. Such system may include (1) a first monitor wirelessly receiving video transmissions in real time at a first frequency and a second monitor receiving patient parameter data in real time at a second frequency different than the first frequency of the video transmissions; (2) a first monitor receiving real time video transmissions by a hard-wired system at any desired frequency and a second monitor receiving patient parameter data in real time at any desired second frequency; and (3) a common monitor for receiving wirelessly transmitted real time video transmissions at a first frequency and also receiving patient parameter data in real time at any desired second frequency. The video image can be stored locally so that it is retained in the case of a power failure.

Thus, with the present inventive system, an existing hardwired LAN (local area network) such as in a hospital or other structure can be used to locate a person having a transponder associated therewith that is in wireless communication with a LAN node in the building or structure and instructions may be sent by the doctor to appropriate personnel, to the doctor for informational purposes, such as patient location, or commands may be sent to equipment such as the patient's transponder to cause it to change its operating mode such as, for example only, to increase the rate of monitoring or the like.

The parameter sensors, or transducers, are of the miniature type that can be affixed to, or inserted in, the physical body by well known means such as suction cups, adhesives, surgery, and the like and are provided with a snap, or other well-known connectors, to which the electronic unit of the transponder can be removably detached. The electronic unit of the transponder may be a well-known type such as an R/T unit with the trademark SmartRF®, part number CC1020 manufactured by Chipcon, or an R/T unit manufactured by RFMonolithics, Inc., part number 0001. In the preferred embodiment, it includes a transmitter, a receiver, and a microcomputer having a measured parameter analog signal receiving section, a conversion section to convert analog signals to digital signals, a memory for storing the converted digital signals, necessary timers for allowing the transponder to sleep for predetermined time intervals to conserve battery power (if an internal battery is required), a transmitter for enabling transmission of stored data at regular intervals, or upon interrogation or upon request from a remote receiver, and a receiver section for receiving interrogation signals or other command signals for necessary computer operation. The transmitter is able to transmit at any one of three different frequencies; 900 MHz, 2.4 GHz, and 5.8 GHz. Further, the entire transducer/transponder combination can be miniaturized and formed as a single unit for use such as an emplacement device surgically implanted within an animate body.

Thus, the primary functions of the transponder are to detect measured physiological, and/or biological, parameter signals, in analog form, convert the analog signals to digital data signals, process and store the digital data signals when necessary, and transmit the detected parameter signals to a remote receiver where they can be analyzed by competent personnel. As stated earlier, the measured parameters may be of any type such as, but not limited to, blood pressure, blood sugar, oxygen use, heart beat rate, electrocardiogram signals, and moisture generation.

A secondary function of the transponder is to receive commands from a remote device such as a receiver/transmitter/monitoring device that, inter alia, performs functions such as, but not limited to, transmitting interrogation signals, timing changes, ID code assignments, and changes to various sleep modes, alarm variables, and other system parameters to the transponder.

As stated earlier, the primary function of the remote device is to receive transmitted physiological and/or biological parameter data from the transponder and provide the data to a monitor where the parameters can be reviewed by competent personnel. Further, the remote receiver may be used to provide an interface with a portable user device such as a PDA, a laptop PC, PAGER, or any other type of wireless display device. This user device may also present the monitored parameters in graphic text form for viewing by the nurse, doctor, or other qualified personnel. Either the remote receiver or the user device may provide data such as the identification code and address of the physical body whose parameters are being monitored so that, for instance, a doctor or ambulance personnel can be directed to the appropriate patient location. Also, as stated earlier, the remote device may be used to provide any necessary authorization, authentication, pre-analysis, and the like information to the transponder. Further, the remote receiver can be in the form of a basic PCMCIA adapter card that operates at a given radio frequency such as 2.4 or 5.8 GHz. That frequency could be changed to accommodate existing RF devices that operate at any given frequency by providing a CF (compact flash) card that plugs into the PCMCIA card and causes the transmitter to transmit an RF signal at a different frequency such as, for example only, 900 MHz.

Of course, the transponder microcomputer can be programmed as an OEM device during manufacture, or the program can be modified in existing equipment, to perform necessary functions with the use of software. Such software defined operation, either OEM or for the aftermarket, would allow interfacing with existing networks to provide increased levels of security by having unique software security routines without any changes to existing interface standards.

The video image system preferably uses a video camera whose images can be transmitted, either by hard-wire or wirelessly, at a frequency the same as, or different from, the patient parameter transmission frequency (e.g. 2.4 GHz, 5.8 GHz, or 900 MHz) to a remote location for viewing. The video imaging system can be used with the hardwired system disclosed herein either by transmitting directly to the access units of the hardwired system at a frequency different from the patient parameter transmission frequency and using a common monitor for both the video viewing and the patient parameter viewing, or hardwired to a separate monitor from the patient parameter viewing monitor and transmitting at any desired frequency, or by transmitting directly to the access units of the hardwired system a frequency different from the patient parameter transmission frequency and using a separate monitor to view the video transmission separately from the patient parameter monitor.

Thus, it is an object of the present invention to provide a unique wireless physiological parameter measuring system using a transponder that measures the vital statistics of a physical body and transmits those vital statistics, in real time, to a remote monitoring receiver without any direct physical connections between the transponder and the remote monitoring receiver.

It is another object of the present invention to provide a unique wireless physiological parameter measuring system forming a Local Positioning System (LPS) that utilizes a hardwired LAN network, WLAN (Wireless LAN), or WWAN (Wireless Wide Area Network) to inform the personnel receiving the transmitted physiological parameters at a remote location of, not only the measured physiological parameters but also the exact location of a person, patient, or physical object at any address or in a room within a building such as a hospital. It is to be understood that the exact location of a person, patient, or physical object at any address or in a room within a building can be determined with the use of well-known satellite positioning systems in conjunction with the Local Area Network (LAN).

It is still another object of the present invention to provide a unique wireless physiological parameter measuring system that utilizes a RF wireless transponder that is removably attached to a parameter detecting transducer that is affixed to the physical body.

It is yet another object of the present invention to provide a unique wireless physiological parameter measuring system that enables a rapid response to an emergency health condition of a person by enabling the nurse, doctor, or other qualified personnel to carry the remote receiver in the form of various devices such as, but not limited to, a PC tablet, Pager, or a PDA. When detected physiological parameters indicate an emergency, the parameter data, along with a warning alarm and patient location, is transmitted to the PC tablet, PDA, Pager, or other remote receiving device and displayed for the benefit of the qualified personnel.

It is also an object of the present invention to provide a unique wireless physiological parameter measuring system that is enabled to connect to various existing hardwired LAN systems in, for example, a hospital or nursing home and that operate at different RF frequencies. The remote receiver, in the form of a PC, PC tablet, PDA, Pager, or other RF receiver device, can use a plug-in card to change the operating frequency of the remote receiver to match the existing system operating frequency thereby enabling the novel system to be frequency compatible with other existing hardwired system frequencies.

Thus, the invention relates to a wireless physiological parameter measuring system comprising at least one transponder associated with a human body whose physiological parameters are being measured and including at least one transducer for generating signals representing the measured parameters, and a circuit for receiving the measured parameter signals from the transducer, converting the signals to transmittable signals, and wirelessly transmitting the measured parameter signals, and at least one remotely located receiver for receiving the wirelessly transmitted measured parameter signals from the transponder.

The invention also relates to a wireless physiological and/or biological parameter measuring system wherein a plurality of remotely located receiver units are spaced at locations such that at least one of the receiver units is in wireless communication with the transponder at all times.

### BRIEF DESCRIPTION OF THE DRAWINGS

These and other more specific objects of the invention will be disclosed when taken in conjunction with the following detailed description of the drawings in which like numerals represent like elements and in which:

FIG. 1 is a schematic drawing representing a first embodiment of the invention wherein a transponder is associated with the body of a person such as an athlete or a patient and that wirelessly transmits measured physiological parameters of the person to a remotely located signal receiver, preferably RF, and from thence to a display monitor unit to allow qualified personnel at said remote location to analyze said measured and displayed parameter signals in real time;

FIG. 2 is a schematic drawing representing a second embodiment of the invention wherein a transponder is associated with the body of a person such as an athlete or patient that wirelessly transmits measured physiological parameters of the person to a remotely located RF signal receiver and from thence to at least one node of an RF signal Local Area Network (LAN) that carries the RF signals by hardwired connections to at least one other node of the LAN where the RF signals are received by a second RF signal R/T unit where the RF receiver is associated with a display unit to enable the measured physiological parameters of the person to be analyzed and/or monitored by qualified personal and where the transmitter unit wirelessly transmits the measured parameters, or signals associated therewith such as warning signals, patient identity, patient room number and the like, to a portable RF receiver unit such as a PDA, laptop computer, or the like to notify competent medical personnel of a patients condition and the patients location as well as to the transponder to request retransmission of the signal parameter of the received signal that does not meet predetermined transmission requirements;

FIG. 3 is a schematic drawing of a third embodiment of the present invention in which a transponder is associated with the body of a person such as an athlete or patient and the transponder transmits physiological parameters of the person to a remote RF signal receiver and from thence to an RF signal transmitter that is in signal communication with a second, more remotely located, RF signal receiver and associated display monitor located at great distances from the person by means of a WWAN system including a satellite system, internet system, or terrestrial wireless system;

FIG. 4 is a block diagram of the novel transponder that is associated with the body of a person such as an athlete or patient;

FIGS. 5A, 5B, and 5C are schematic mechanical and electrical diagrams illustrating the physical relationship and operation of the transponder components;

FIG. 6 is a block diagram of a prior art integrated radio chip set that can be used as the 2.4/5.8 GHz RF receiver/transmitter unit;

FIG. 7A is a schematic diagram of a 2.4/5.8 GHz radio adapter that plugs in to an RF receiver/transmitter access unit and is used to cause the RF receiver/transmitter access unit to operate at 2.4/5.8 GHz;

FIG. 7B is a schematic diagram of a 900 MHz radio adapter that plugs into the 2.4/5.8 GHz radio adapter to enable the RF receiver/transmitter access units to operate at 900 MHz in surroundings where radio interference may exist or where the existing hardwired LAN systems operate at 900 MHz;

FIG. 8 is a schematic drawing of a hardwired LAN system used, for instance, in a structure such as a hospital, illustrating how the physiological parameters of a person, located in one room of the hospital and having an associated transponder that is transmitting physiological information of the person, can be received by the existing LAN system and recovered in any other remotely located room of the building having a LAN terminal therein;

FIG. 9 is a schematic representation of another embodiment of the invention in which an individual having multiple associated transponders, each of which measures a particular body parameter and each of which transmits the measured parameter data to an RF access unit located on the person of the individual where the parameter data is processed and then transmitted to a remote RF access unit for display and analysis. Where multiple body parameters are being measured and transmitted, they may be all transmitted at the same frequency, in which case an analog switch is used to select which of the parameters is being transmitted. This analog switch may be controlled from software in the base-band processor associated with the receiver inasmuch as the attending physician or other competent personnel may wish to view a particular parameter that is being measured and transmitted.

The multiple parameters being measured may also be transmitted on different frequencies. In such case, the receiver may have filters, well-known in the art that can select any one of the frequencies for reception thus allowing the personnel monitoring the parameters to select the parameter they desire to review.

FIG. 10A is a schematic representation of a hard-wired video imaging system having a first monitor for video viewing at any given frequency and used in conjunction with a second monitor for the patient parameter viewing at any given frequency;

FIG. 10B is a schematic block drawing illustrating the use of a common monitor to view both the video images transmitted at a first frequency and the patient parameter data transmitted at a second different frequency; and

FIG. 10C is a schematic block drawing illustrating the use of a first monitor for viewing the video images transmitted at a first frequency and a second separate monitor for viewing the patient parameter data transmitted at a second frequency different than the first frequency.

### DETAILED DESCRIPTION OF THE DRAWINGS

For purposes of simplicity, the invention will be described herein in relation to a person such as an athlete or a patient. However, it should be understood that the invention, when appropriately modified, could also be used with inanimate systems such as measuring water purity, tracking weather conditions, and the like.

It is well known that in certain cases, athletes have suffered severe body damage, and even death, while playing in an event such as basketball, football, soccer, and the like. If the physiological parameters of the athlete could have been monitored in real time, competent personnel could have noted the physiological changes occurring in the athlete, either visibly or by means of an alarm, before the damage was extensive and may perhaps have even avoided the death of, or serious injury to, the athlete. In like manner, a patient in a hospital operating room has to have physiological parameters (such as blood pressure, temperature, heart rate, respiration, and the like) monitored in real time during surgery. At present this requires cables to be physically connected between the patient and the monitoring devices. These cables create difficulties for the surgeons who must move around and over them during the surgery.

Further, patients recovering in hospital rooms such as IC, patient rooms, and the like have monitoring devices physically coupled to them with cables and the like so that nurses at the nursing station can monitor the patient's physiological parameters in real time. Alarms can be sounded when the physiological parameters exceed or are outside of preset limits. Such cables limit movement of the nurse, or other medical personnel, to an area in the immediate vicinity of the monitor because, if the nurse moves away from the monitor for any reason, an emergency signal may be received from a patient that is not seen by the nurse, thus enabling unnoticed adverse conditions to exist for the patient.

The present invention enables real time monitoring of desired physiological parameters of athletes, patients, and others when sudden emergencies may occur without the use of tethers, cables, or other physical connections that limit movement of the athlete or patient and that limit the mobility of the personnel who monitor the patient or the athlete.

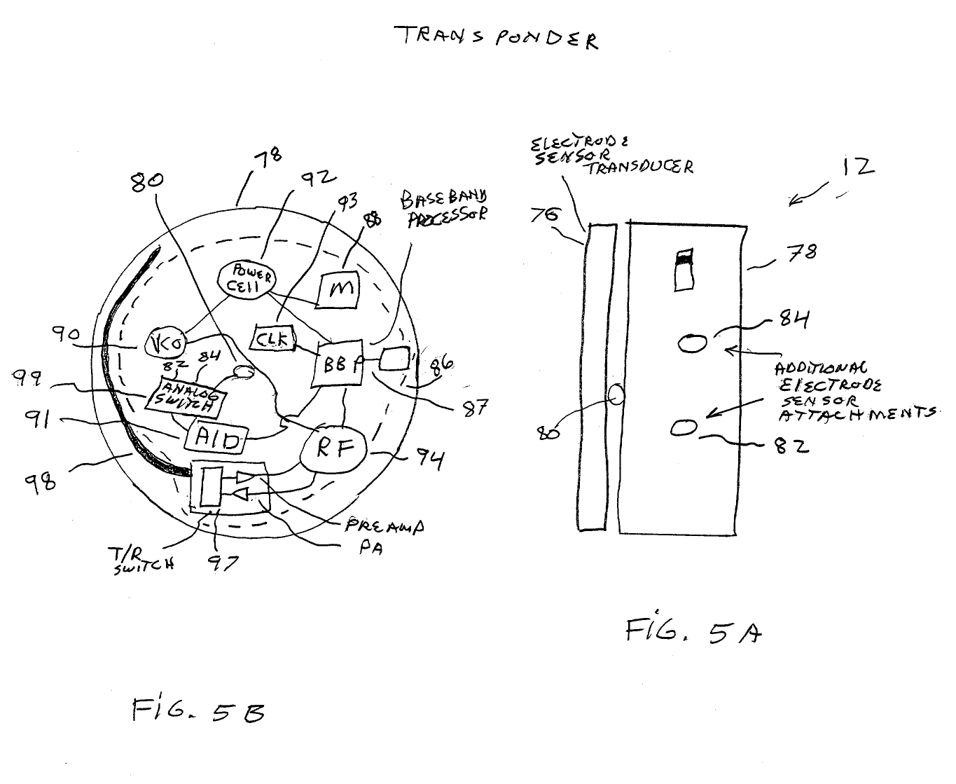

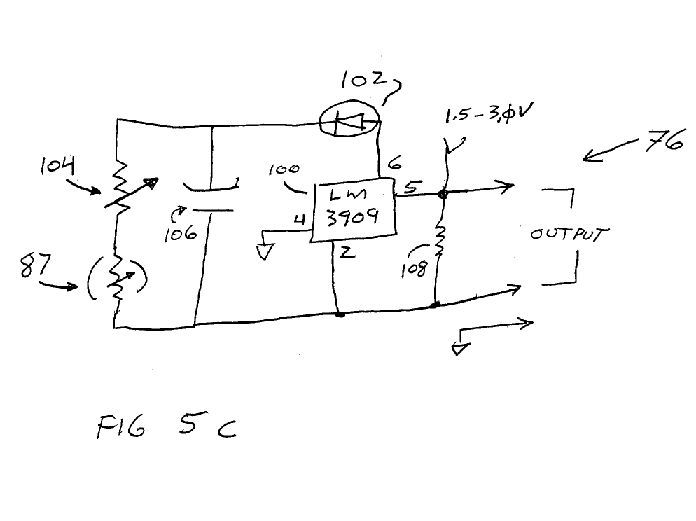

FIG. 1 is a schematic diagram of a first embodiment of the present invention in which a person 10 (herein defined as a patient, athlete, or any other category of person) has a transponder 12 associated with or attached to the person in any well know manner such as by adhesive, implantation, or the like. The transponder 12, as will be more fully described hereafter, includes (1) a transducer 76 (in FIG. 4) that, in the preferred embodiment, is detachable from the transponder 12 and is used for measuring at least one physiological parameter such as blood pressure, heart beat rate, blood sugar, body temperature, and the like, and (2) an electronic unit 78 (in FIG. 4) including an A/D converter for converting the analog parameter to a digital signal, and a receiver/transmitter unit for wirelessly transmitting the signal data, preferably at RF frequencies, to a remotely located RF receiver 14. The transmitted parameter signal will be 2.4/5.8 GHz or 900 MHz. The RF receiver 14 couples the received parameter signal data through medium 16 (telephone wire, cable, and the like) to a monitor 18 where the parameter can be reviewed on a display monitor by competent personnel. This system may be used, for example only, by an athlete on an outside track, golf course, basketball court, football field, and the like, or by a patient in a room in a hospital, nursing home, or other structure. An athlete, for instance, may be running on a track, or playing on a football field, or playing on a basketball court. In each of these examples, multiple receivers 14 may be placed at spaced intervals around the track, the field, or the court such that, in any position on the court, the field, or the track, at least one receiver 14 will receive the wirelessly transmitted physiological parameters that are being transmitted from a transponder 12 on the athlete. Each transponder 12, associated with a person 10, may have an identification code associated with the data transmission such as a header or can be located at any other position within the data transmission. By knowing which transponder is associated with each person, competent and relevant personnel monitoring the transmissions identify each individual person, his or her location, and his or her physiological parameters as they are being received in real time.

For a patient 10 in a hospital room, the transponder 12 wirelessly transmits the patient's desired physiological parameters to receiver 14 that is generally in the same room with the patient and is coupled to a display monitor 18. Thus, during surgery, for example, the desired patient physiological parameters are transmitted wirelessly to the receiver 14. The display monitor 18, coupled by medium 16 to the receiver 14, can be used in the operating room by relevant medical personnel to monitor the patient's vital signs during the surgery without the use of cables or any other physical connections between the patient and the monitoring device. Thus there are no cables to interfere with the movements of the surgeon(s).

It can, therefore, be seen that the inventive system of FIG. 1 will allow the physiological parameters of an active athlete to be monitored in real time during the athletic event without any physical cables coupling the athlete to the monitor and, while allowing the athlete to participate in the event, may prevent injury to, or even death of, the athlete by observing the physiological parameters as they are occurring in real time therefore alerting competent medical personnel who are monitoring the physiological parameters during the actual occurrence of the athletic event to any medical emergency or simply to electronically store or record the measured parameters for future evaluation and use.

As stated earlier, the same monitoring takes place in the operating room of a hospital where, during actual surgery, in real time, the patient's vital signs are visually monitored without the requirement of cables being physically attached to the patient. This is a great advantage to the surgeons and physicians.

The system is extremely useful in other situations involving the health of a patient. When a patient has a transponder 12 associated with his/her body that is measuring physiological parameters, the measured parameters may be transmitted to any competent medical personnel at any location such as the doctor's office, any hospital room, ER, ICU, and other areas where there is located a receiver 14/monitor 18 system as shown in FIG. 1.

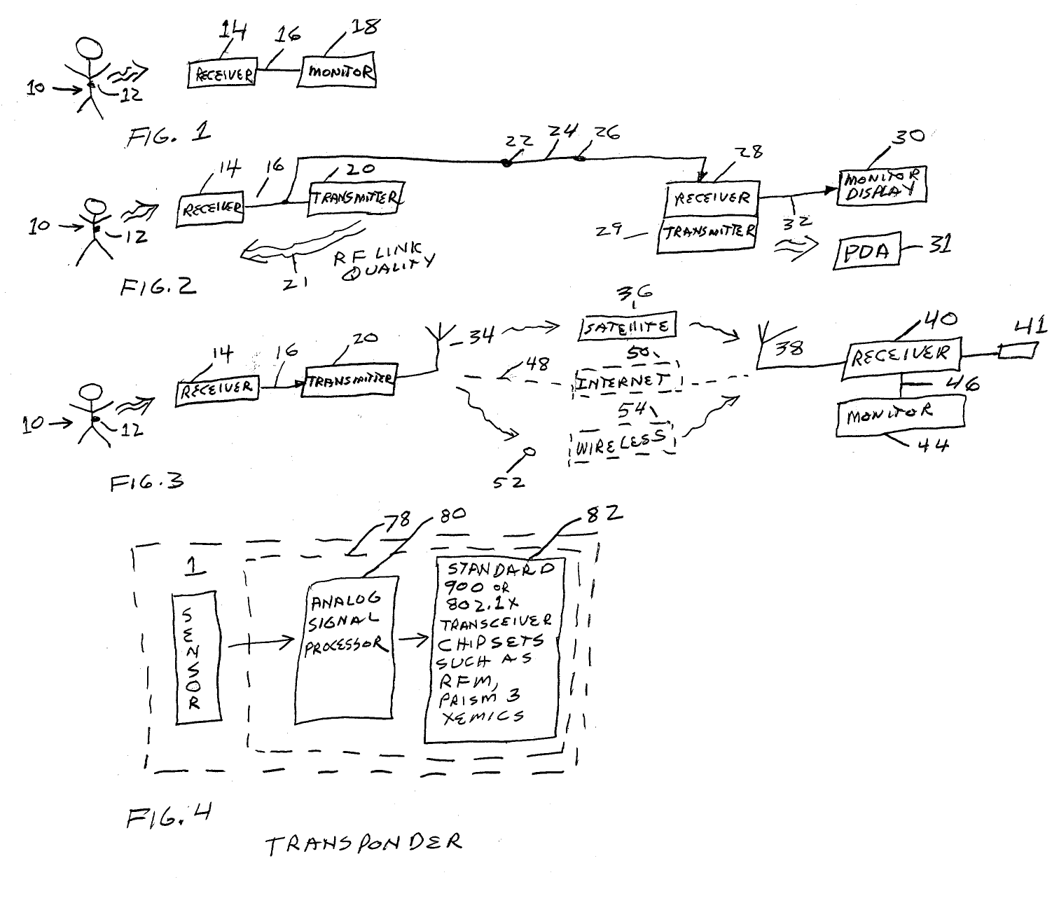

FIG. 2 illustrates the system in use with a Local Area Network (LAN) such as may be installed in or pre-exist on site in a hospital or other such structure. The details will be shown in relation to FIG. 8 but the principles are shown in FIG. 2. The same system is used as shown in FIG. 1 except that a receiver 14/transmitter 20 is coupled together at 16. The receiver 14 is directly connected to node 22 to send the received physiological parameter data to all of the other parallel nodes 26 of an existing LAN system 24. The LAN system 24 may be in, for instance, a structure such as a hospital or a nursing home. The hardwired LAN system 24 couples in parallel a multiplicity of connected nodes, such as 22 and 26. Each of the other parallel connected nodes 26 may be located in other rooms in the structure. The receiver/transmitters 14/20 and 28/30 operate at a given frequency. As stated, they are generally coupled in parallel to the hardwired system 24 within the hospital or other structure such as a nursing home. Transmitter 20 receives the measured parameter signals from receiver 14 and, first, transmits control signals at 21 (i.e. “retransmit signals”) back to the RF receiver circuit forming part of the transponder 12 for quality control purposes such as RF link quality. Thus, if the signal quality is not within predetermined acceptable parameters, the transponder 12 is asked to retransmit the signal to try to obtain better quality. Second, as will be discussed hereafter, the signal from transmitter 20, transmitted at path 21, can also be received by portable monitoring devices such as a PDA, a laptop computer, a Pager, and the like so that a nurse, doctor, or other competent medical personnel may not be required to stay at a permanent station but can move around while maintaining the ability to monitor patients.

The receiver 28/transmitter 29, at receiving node 26, couples the received parameter signals to a monitor 30 via connection 32. The receiver 28 and monitor 30 may be located at a nurse's station, a doctor's office, or any other pertinent location. Thus, the patient, in this case, may be located in one room of the structure and the patient's physiological parameters monitored in another area of the structure. This will be seen more clearly in relation to the discussion of FIG. 8 that may represent, for example only, a hospital or a nursing home 56 having multiple rooms 58-74 therein, each of which has an R/T node 22. Room 58 may be an operating room in a hospital 56. Room 60 may be the cafeteria. Room 62 may be the rehab room. Room 64 may be a patient room. Room 66 may be a recovery room. Room 68 may be the ICU room. Room 70 may be the security room. Room 72 may be the admissions/discharge room and room 74 may be a maintenance room such as a record maintenance room wherein is located a server 75 that not only maintains a record of the patients medical history but also can change the operating parameters of the LAN system (and the transponder 12) such as frequency of monitoring patient parameters or which parameters are to be measured where multiple patient parameters are being monitored.

Other rooms wherein R/T nodes 22 could be located could, of course, include the nurse's station, doctor's offices, and the like.

It will be noted that a node in each of these rooms, such as node 22, includes a wireless receiver 28/transmitter 29 (R/T) therein as mentioned earlier. Thus, the physiological parameters of the person being monitored are transferred to all of the nodes 22 in the structure and are then transmitted by transmitter 20 (or 29 in FIG. 2), first, to the transponder 12 receiver for RF link quality maintenance. Second, the RF signal is transmitted to any receiver in the receiving vicinity of the transmitter 20/29 at any node 22/26. This means that if a nurse, doctor, or other competent medical personnel has left an assigned station, they can carry with them a portable receiving device 31 as shown in FIG. 2, such as, for example, a PDA, a lap top computer, a pager, or other wireless receiving device and receive an alert signal with the portable receiving device concerning a particular patient or person whose physiological parameters are being monitored. The alert signal may be generated either audibly and/or visually by a display monitor at the receiving device. The medical personnel can then access, on the display monitor forming a part of the receiving device, the real time measured physiological parameter or parameters of the person and see the parameter (in graphic, numerical or other form) and the location of the patient or person so that immediate attention can be given to the person. It is understood that the prior art utilizes RFID tag technology with clothing, department stores, and warehouses for theft prevention by identifying and tracking materials and their movement. The present invention, however, is novel in that it provides real time monitoring of actual measured continuing physiological parameters of a person over an actual time period as they actually occur such as monitoring certain parameters during surgery or monitoring other parameters during a physical performance of a person such as an athlete.

In FIG. 8, it will be noted that in the operating room 58, the measured parameters are transmitted, not only to the R/T at node 22 but also is transmitted to a combination receiver/monitor 19 in the confines of the operating room 58 where the patient's physiological parameters may be observed during surgery in real time by a person in the operating room. Of course they could also be monitored remotely through the hardwired LAN system in some other room as explained earlier.

In FIG. 3, a third embodiment of the present invention is shown. This system operates similar to the first and second embodiments except that the measured physiological parameters can be monitored off the patient site to more remote locations such as in another city, hospital, nursing home, doctor's office, or the like with the use of satellite, the internet, a WLAN, a WWAN, or the like. The receiver data base may contain software that compares the received parameter data with stored reference data that is constantly updated. Such updated reference data can relate to medical science or medical issues regarding the patient/athlete parameters being monitored.

In FIG. 3, the transponder 12, attached to the person 10, again transmits the real time measured physiological parameters to a receiver 14 and thence by connection 16 to transmitter 20. Transmitter 20 not only radiates the signals back to the receiver in transponder 12 for RF signal link monitoring as explained earlier, but also radiates from antenna 34 to either a satellite 36, wireless connection 50 such as a WWAN, WLAN, LAN or internet connection via signal path 48, or through a terrestrial wireless network 54 (other than satellite) via transmission path 52. In all three transmission paths, the signal is received by antenna 38 that is coupled to receiver 40/transmitter 41. Receiver 40 is further connected by signal path 46 to a monitor 44 where the physiological/biological data parameters can be monitored at the remote location. This embodiment enables the data parameters of the person being measured to be transmitted to medical experts, or other experts in a given field at remote locations, off patient site, where diagnoses or specialized treatment for the given person may be prescribed even though such experts are not located on site where the person or patient is located.

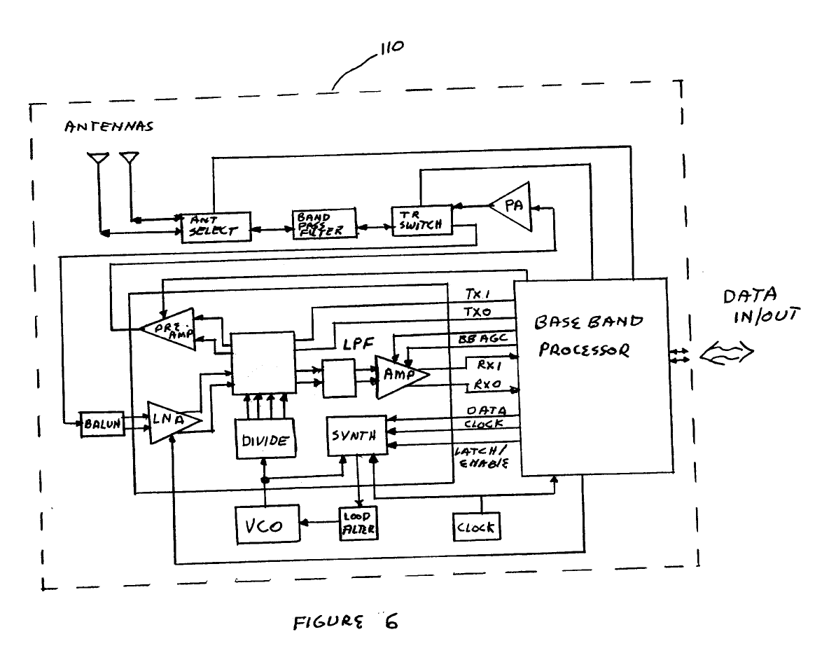

FIG. 4 is a schematic block diagram of the transponder 12. It has two major components. The first component is the sensor 76 that is attached in any well known manner to the person or patient and that measures physiological or biological parameters. The second component is the electronics 78 comprising the signal processor 80 and the receiver/transmitter 82. The sensor 76 will be described in detail in connection with FIG. 5C and connects the measured analog parameter to the analog signal processing unit 80 where the signal is processed and converted from an analog to a digital signal. With miniaturization, some signal pre-processing could be performed in the sensor 76 in a well-known fashion as will be described hereafter in the discussion of FIG. 5C.

From the signal processing unit 80, the digital signal is coupled to receiver/transmitter 82 where the signal can be transmitted wirelessly to a remote receiver/transmitter unit as explained earlier. The receiver/transmitter 82 (R/T) is of a well-known type such as the PRISM³® (a registered trademark of Intersil Americas Inc.). This R/T unit 82 is manufactured to operate at either 2.4 or 5.8 GHz (or 900 MHz to match some existing access units that operate at other frequencies such as at 900 MHz, the frequency of some existing LAN systems). However, existing remote R/T units (such as 14/20 and 28/29 in FIG. 2) may be modified, as explained hereinafter, to operate at a frequency, such as 900 MHz by the use of a plug-in adapter card.

R/T unit 82 transmits the measured parameter data to access units at remote locations as discussed above in relation to FIGS. 2, and 3. The R/T unit 82 also receives command signals to adjust the operating parameters of the transponder 12 in a well known manner.

FIGS. 5A, 5B, and 5C are schematic block diagrams illustrating the components and relationship of the transducer or sensor 76 and the electronics unit 78 of the transponder 12 shown in FIGS. 1-4.

FIG. 5A illustrates the entire transponder 12. It consists of the sensor 76 and the electronics unit 78. The sensor 76 connects to the electronics unit 78 via snap or plug type connectors 80. This forms an integrated package that can be applied directly to the individual and can also accept additional inputs 80 and 82 from other remotely located sensors on the body of the individual with the use of short cables. In a surface skin temperature application, the sensor 76, as shown in FIG. 5C, contains a temperature sensing device 87 embedded into a flexible package that forms the sensor or transducer 76. This sensing device 87 is similar to standard electrode patches now in use. The sensor 76 removably mates via snap plug/socket connection 80 to the circuit electronics 86 that includes the R/T elements. While only one snap plug/socket connection 80 is shown in FIG. 5B, it is clear from FIG. 5A that more than one socket connection 82/84 can be used so that multiple physiological measurements may be taken simultaneously. In transducer 78, as shown in FIG. 5B, base-band processor 87 selects which processing circuit (i.e. heart beat rate, temperature, and the like) should transmit a signal. For instance, a software program installed in the base-band processor 87 might tell the transponder 78 to transmit heart beat rate signals every 30 seconds, and to transmit temperature data signals interleaved between the spaced heart beat rate transmissions. The software program in the base-band processor can thus control an analog switch in the transponder that switches between the measured temperature parameter signals and the measured heart beat rate parameter signals as described earlier. Thus, the temperature processing circuit can be deactivated with the analog switch when the heart beat rate processing circuit is energized and when the temperature processing circuit is energized, the heart beat rate circuit can be deactivated by analog switch in accordance with the software instructions.

The T/R circuit electronics 78 shown in FIG. 5B includes a microprocessor 86 with a memory 88, the necessary clock or clocks 97, the A/D converter 91, and the VCO 90. A power cell 92 may be included although power may be coupled to the electronic unit 78 externally with a command signal as is well known in the art. The microprocessor 90 causes the measured physiological parameter(s) to be stored in memory 88 and to be transmitted from RF circuits 94 by means of a R/T switch 97 for transmission through transmit/receive circuits 96 to antenna 98. Obviously, command signals from a remote interrogation unit (R/T) would be received through the R/T switch 97. The output of the sensor or transducer 76 on snap electrode 80 is interfaced with the R/T circuit, or electronic unit, 78 as shown in FIG. 5A. The sensor 76 will function with an R/T circuit, or electronic unit, 78 that operates at any frequency; i.e. 900 MHz, 2.4 GHz, or 5.8 GHz. As stated previously, the sensor 76, (attached to the body as indicated), could be connected to the R/T circuit, or electronic unit, 78 with a small cable for multiple physiological parameter measurements. An LED (not shown) could be placed in the R/T circuit, or electronic unit 78, instead of the sensor 76, to give a visual indication that the device is operating.

FIG. 5C is a circuit diagram of a sensor or transducer 76 for measuring body temperature. The basic circuit includes a LM 3909 microchip with an output signal being generated between pins 2/5 across load resistor 108. An LED 102 may be connected to pin 6 of the LM 3909 chip to give a visual indication that the circuit is operating. If desired, the LED 102 may be a part of the R/T electronics unit 78 as shown in FIGS. 5A or 5B, as stated earlier, instead of the sensor or transducer 76. The temperature sensing element 87, a thermistor, for example only, senses the body heat and changes resistance accordingly. The sensor 76 may be calibrated with variable resistor 104. The analog voltage signal generated by the changing resistance of the thermistor 87 is stored in capacitor 106. Power may be supplied to the sensor 76 circuitry by means of a power source coupled to pin 5 of the LM 3909. Of course the power source may be located either in the sensor 76 or the R/T electronics unit 78. A ground terminal from pin 4 may also be coupled to the R/T electronic unit 78. Some signal processing, such as signal amplitude adjustment, can take place in the sensor 76. Of course, different sensing elements would be used for each physiological parameter being measured. Such sensing elements include, but are not limited to, piezoelectric devices, magnetic sensors, and the like.

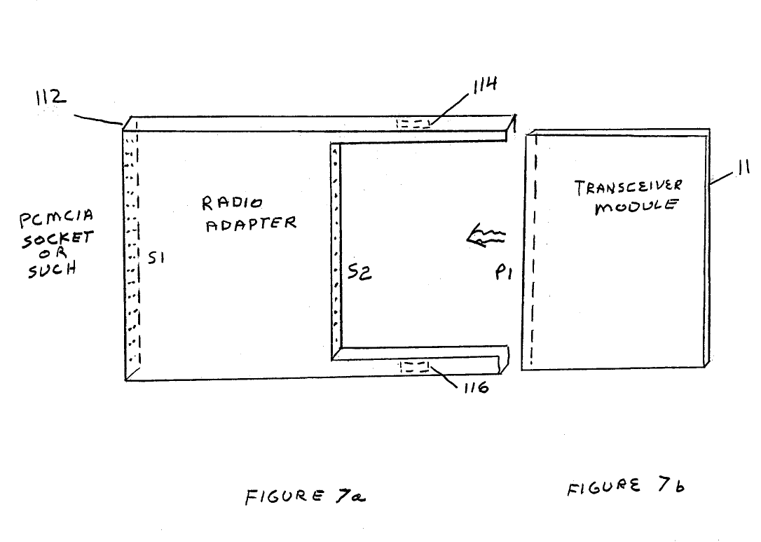

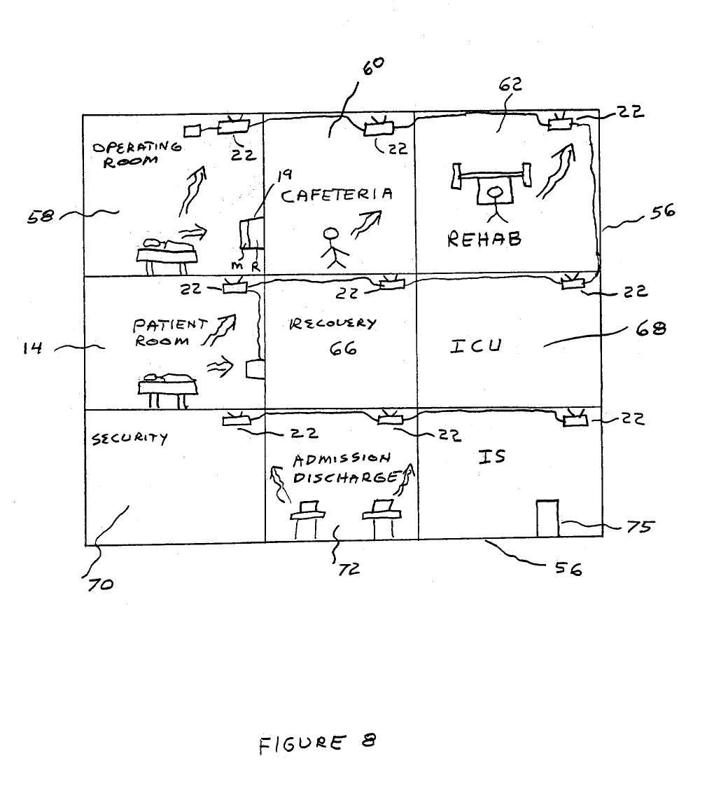

FIG. 6 discloses a schematic diagram of an existing 2.4/5.8 GHz integrated chip set 110. This circuit can be utilized as the transponder 12 in FIGS. 1-3 and obviously can be modified to operate at a frequency of 900 MHz if necessary. As stated previously, it may be of the type known as the PRISM®3 that is manufactured by Intersil®. Inasmuch as this chip set is commercially available, no discussion of the chip circuitry will be presented here. This circuit can be formed as a plug-in adapter 112, shown in FIG. 7A, that can be inserted in a PCMCIA socket of an existing hardwired electronics unit at nodes 22 (shown in FIG. 8) to cause the hardwired electronics to operate at 2.4/5.8 GHz. Antennae 114 and 116 (FIG. 7A) provide a 2.4/5.8 GHz wireless connection to a WLAN or air interface.

If the operating frequency of an existing hard-wired system is 900 MHz, a second plug-in card 118 is inserted with pins P1 into socket S2 of first plug-in card 112 as shown in FIG. 7A and FIG. 7B. The separation or mating of the socket S2 and pins PI provide an enabling or disabling of the 2.4/5.8 GHz operating frequency of the hard-wired access electronics unit 22 and the disabling or enabling of the 900 MHz frequency by means of second plug-in card 114.

If it is desired to operate at either 2.4/5.8 GHz, and more memory storage is needed, the second plug-in card 118 may be a memory expansion unit instead of a frequency changing unit.

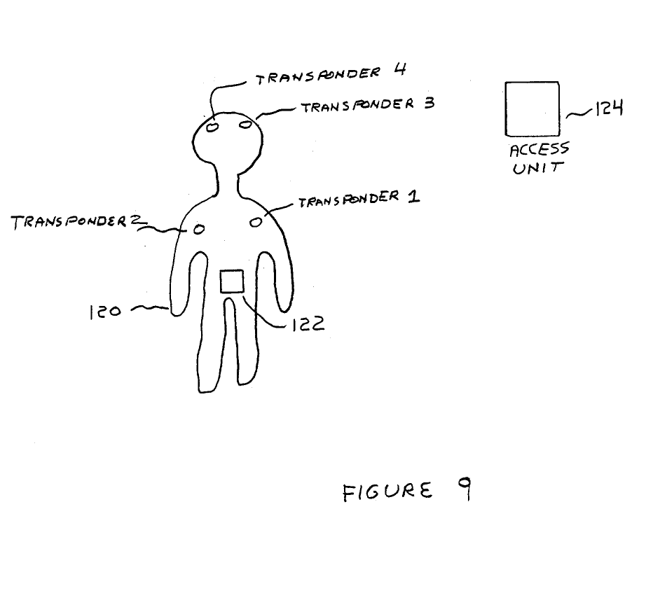

FIG. 9 is a diagrammatic representation of another embodiment of the present invention in which multiple transponders 1, 2, 3, and 4 are associated with the body of a person 120 and wirelessly transmit measured physiological and/or biological parameter data to a first R/T access unit 122 on the person whose physiological parameters are being measured. There the measured parameter data can be processed and stored until it is time to transmit such parameter data to a remote R/T access unit 124 in a manner as previously described. Each of the transponders 1-4 may have an ID code associated with each transmission so that particular transponder transmissions can be identified as received from a particular transponder.

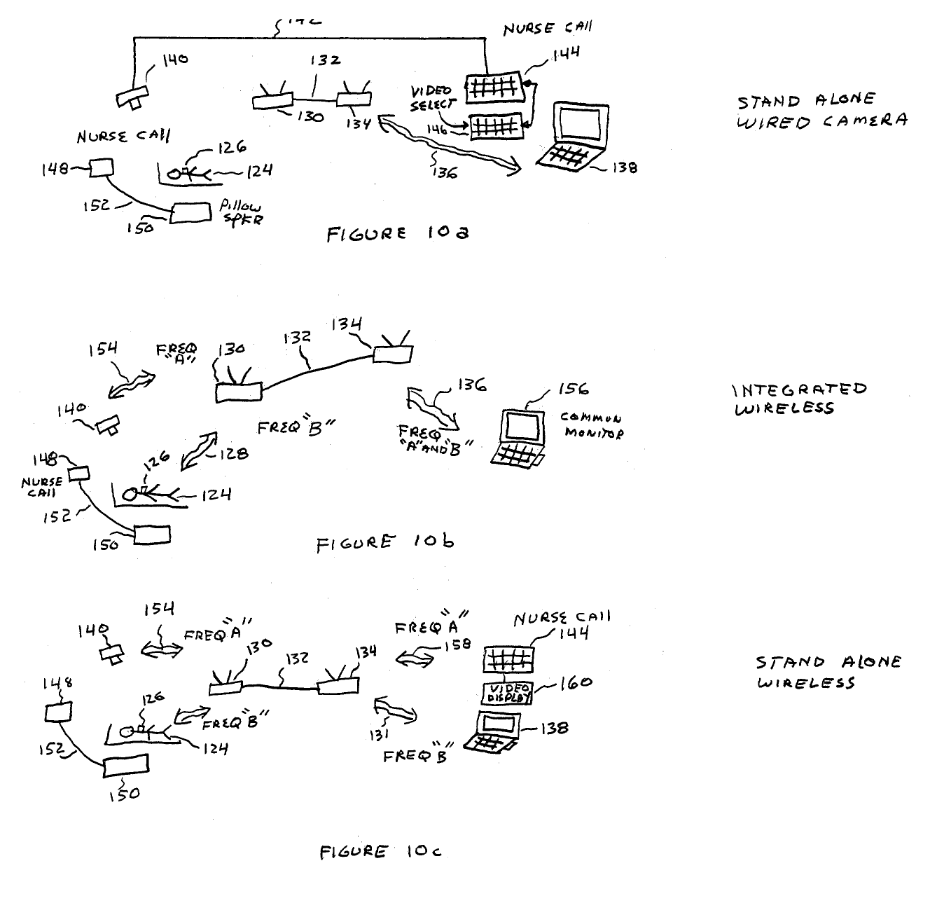

FIG. 10A is a schematic block diagram of a novel system for adding video image viewing of the patient in real time. As can be seen, the patient 124 has the transponder 126 attached in a manner previously described. The transponder 126 transmits at 128 to access unit 130 of a hard-wired existing system 132 for transmission to a plurality of parallel nodes 134. At node 134, as described previously herein, the patient parameter data is transmitted through path 136 to a remotely located monitor 138 which may be of any of the types described earlier herein. In addition, a video camera 140 may be positioned so as to take real time video images of the patient when the video camera 140 is selectively activated in any well known manner. When activated, the real time video images are transmitted along hard wired path 142 to a second monitor remotely located monitor 144 for viewing the patient in real time while simultaneously monitoring the vital statistic parameter data of the patient. In this system, the video image transmission can by of any desired frequency since that signal is passed over a hard-wired path 142 separate from the transmission of the patient parameter data. This is an advantage of using this type of system.

FIG. 10B is a schematic block diagram of a similar system to that shown in FIG. 10A except that the existing hardwired system 132 carries both the patient parameter data as well as the video imaging data. In this case, however, the transmission frequency of the video images must be different from the transmission frequency of the patient parameter data signals. Thus, the patient transponder 126 again wirelessly transmits patient parameter data at a first frequency along path 128 to access unit 130 of the hardwired system 132. That data is transferred to any of the parallel nodes 134 and is then wirelessly transmitted along path 136 to a remotely located monitor 156 that may be at a nurse's station or other point in the system as described earlier herein. In addition, the video camera 140, when selectively activated, transmits video images at a second frequency different from the first frequency along path 154 to access unit 130 of the hardwired system 132. That data is, again, transmitted along path 138 at the second different frequency to the remote monitor 156 which is a common monitor for both the video image transmitted signal and the patient parameter transmitted signal. These frequencies have to be different because they use the same transmission paths. In the common monitor 156, frequency selective filters can be used in a well known manner to separate and view the signals simultaneously.

FIG. 10C is like FIG. 10B except that a second separate monitor 160 is used for the video image transmissions that are transmitted from camera 140 along path 154 to the hardwired system 132 as explained with relation to FIG. 10B or to a satellite or other unit, as explained earlier herein, and then along path 158 to the separate monitor 160. In this case, the frequencies need to be different from each other inasmuch as they may indeed use the same transmission network.

In all of the above systems shown in FIGS. 10A, B, and C, there is shown a typical existing nurse call button 148 and pillow speaker 150 coupled to the existing system by conductor 152. It is shown simply because it exists in most hospital systems at present although it has nothing to do with the present invention.

Thus, it can be seen that the novel wireless physiological and/or biological parameter measuring system collects body parameters such as heart beat rate, blood pressure, body mass index, moisture content, and the like. These parameters are collected by at least one transponder on or within the body of a person and are processed and transmitted on either one of the 900 MHz, 2.4 GHz, or 5.8 GHz license free “Industrial, Scientific, and Medical” (ISM) frequency bands.

This system can interface or leverage any existing ISM band infrastructure and can be configured for special frequencies. The system can be overlaid or interleaved with other wired or wireless networks. Range can be up to 75 meters depending on the environment and can increase to several hundred meters or more. Since the transponder in the system is miniaturized, it weighs under one ounce and uses miniature alkaline or lithium ion batteries. Because of the unique circuit and design that allows the device to “sleep” for predetermined periods of time, the life of the batteries is extended many times. The transponder can be interfaced to other monitoring systems that do not have the same system parameters and capabilities. Thus, the system can be modified to operate at different frequencies for the purpose of mating with existing systems.

The transponders are very small and the sensors associated therewith can be implanted in or located on a person and transmit data to a remote T/R unit where the measured physiological parameters can be analyzed and/or stored for future analysis. The remote T/R unit may be a desktop computer, laptop computer, Web Tablet, PDA, or any other data collection device that will receive, store, interpret, and format the physiological information. Preferably, the system operates at Radio Frequencies (RF).

The system is preferably controlled with software that reads and interprets the parameter data received and displays it on “user friendly” display screens. This software allows for internet access and database synchronization through either a wireless network or a wired network. The database could obviously be a single user database in which case there will be no need for synchronization. The database will be located in the computer doing the monitoring. When the system is used with a centralized database, then synchronization will occur upon request or in designated time intervals. The data to be synchronized will be any data that the user needs or has assigned to be synchronized. This data can be patient data, client files, or online links and information that is required to be updated frequently.

The software will also cause the storage of data collected through the sensors. The software will contain databases of information that is readily available to the athlete, patient, Trainer, Nurse, or Doctor. The software can cause storage of group or individual information. The software also provides security by necessitating log-on, log-off, authorized user identity, encryption to secure wired and wireless connections, and the like.

As stated, this software can read and interpret the data and display it on “user friendly” screens. The software allows internet access and database synchronization wirelessly or through a wired network. The software has the ability to cause storage of the data collected through the sensors at several different locations such as in the transponder, the remote R/T unit, PDA's, and the like. The system includes a server that contains a database of information that is readily available to an athlete, trainer, nurse, doctor, or to a patient. These databases of information are included in the software and will be updated and upgraded automatically or when the user chooses.

A user interface is in the form of an electronic manual that is in the software so users can quickly search through the help topics. The software allows and recognizes different users that have different access rights to client or patient data within the system.

The much publicized and possibly preventable deaths of professional and student football and basketball players has brought into focus the need for real time monitoring of certain physiological parameters of such persons. With the present inventive system, coaches and trainers have the ability to remotely monitor vital physiological and/or biological data such as heart beat rate, body temperature, oxygen intake, location, distance traveled, and the like in a user-friendly, wireless virtual private network. The present invention is applicable to high school, college, international Olympic federations, and professional sports teams worldwide. These organizations are not only concerned about their player's health but also the impact of liability in case of injury.

In addition to the safety factor, the present invention will allow coaches, trainers and athletes to customize training programs to assist each player in reaching their peak performance.

The present invention also has immediate application in hospitals and cardiac, hospice, rehab, and healthcare facilities. The invention has been developed around 802.11, 802.xx, or similar WLAN architecture that has unlimited bandwidth and has no air time charges.

While the invention has been disclosed in connection with preferred embodiments, it is not intended to limit the scope of the invention to particular methods and apparatus set forth, but, on the contrary, it is intended covers such alternatives, modifications, and equivalents as may be included within the spirit and scope of the invention as defined by the appended claims.

1. A wireless physiological/biological parameter measuring system comprising:
at least one transducer affixed to an animate body such as a patient/athlete for measuring at least one physiological/biological parameter of said body and generating signals representing said at least one physiological/biological parameter;
an electronic unit coupled to each said transducer for receiving said physiological/biological parameter signals from said at least one transducer and wirelessly transmitting said parameter signals at a first frequency;
at least one remote R/T unit for receiving said wirelessly transmitted parameter signals from said electronic units; and
a display monitor unit coupled to said at least one remote R/T unit for visually displaying said parameter signals.

2. The system of claim 1 further comprising:
attachment means for physically attaching said transducer to said animate body; and
said electronic unit being an RF unit removably attached to said transducer.

3. The system of claim 1 wherein:
said transducer and said RF unit are formed in a single unit to create a transponder as an intelligent sensor.

4. The system of claim 3 wherein:
said transponder is embedded under the skin of said animate body.

5. The system of claim 1 wherein said transmitted parameter signals include an additional signal that identifies the transponder transmitting said generated parameter signals thereby identifying the animate body to which said transponder is attached.

6. The system of claim 1 further comprising:
a hardwired LAN system operating at said first frequency;
at least two nodes coupled to said LAN system;
a first one of said remote R/T units being located at one of said nodes for receiving and wirelessly transmitting RF signals to and from said LAN system at said first frequency;
said transponder transmitting said generated physiological/biological parameter signals as RF signals to said first remote R/T unit at said first frequency; and
a second remote R/T unit at another one of said nodes for receiving said RF signals from said hardwired LAN system.

7. The system as in claim 6 wherein said second remote R/T unit comprises at least one of the class of RF signal receivers including, but not limited to, personal digital assistants (PDA's), personal computers (PC's), pagers, and PC tablets.

8. The system as in claim 1 wherein:
at least one of said remote R/T units is portable and can be carried by personnel to monitor in real time said physiological/biological parameter signals remotely from the source of said parameter signals thereby enabling a rapid response to changing conditions of said animate body.

9. The system as in claim 1 further including:
a first plug-in unit enabling said remote R/T unit to communicate with said transponder at said first RF frequency.

10. The system as in claim 9 further comprising:
an existing LAN system that operates at a second frequency different than said first frequency; and
a second plug-in unit for insertion in said first plug-in unit for converting said first frequency to said second LAN frequency thereby enabling said remote RF signal receiver to communicate with said LAN system at said second frequency.

11. The system as in claim 10 further including:
identification signals associated with each R/T unit at each node of said LAN network to enable personnel receiving said RF signals from any node to identify the node of the LAN network that is transmitting the RF signal thereby enabling said personnel to identify the location of the transponder transmitting the physiological parameter signals.

12. A wireless physiological/biological parameter measuring system comprising:
a plurality of transponders affixed to an animate body, each of said transponders including (1) a transducer for sensing at least one physiological/biological parameter of said body and generating an electronic signal representing said at least one sensed parameter and (2) an electronic unit for receiving said physiological/biological parameter signals and wirelessly transmitting said parameter signal at a first frequency;
a signal associated with each wireless transmission to identify each said transponder;
a receiver/transmitter unit located on said human body for receiving each wireless transmission from each of said transponders and retransmitting each of said received wireless transmissions to at least one remote signal receiver; and
a display unit coupled to said at least one remote R/T signal unit for visually displaying said physiological/biological parameter signals.

13. The wireless physiological/biological parameter measuring system of claim 1 further including:
a video imaging system for generating video images of said human body;
a video transmission system for transmitting said video images in real time to a remote location for viewing in conjunction with said transmitted physiological/biological parameters of said animate body; and
a memory at said remote location for storing said received video images.

14. The measuring system of claim 13 wherein said video image transmission is at a frequency different from said first transmission frequency of said physiological/biological parameters of said animate body.

15. The measuring system of claim 13 wherein said video transmission system is a second frequency that is the same as the first transmission frequency of said physiological/biological parameters of said animate body.

16. The measuring system of claim 14 further comprising filter means located at said remotely located monitors for separating said different frequencies for viewing.

17. The measuring system of claim 14 further comprising:
a first monitor for receiving and displaying the animate body physiological/biological parameters; and
a second monitor for receiving and displaying the animate body video images on a second monitor separate from said first monitor.

18. The measuring system of claim 14 comprising a single remotely located monitor for displaying both said animate body video images and said animate body physiological/biological parameters.

19. The measuring system of claim 15 comprising:
a first transmission path for connecting said video image transmission signals directly to a first remotely located monitor; and
a second different transmission path for connecting said patient physiological/biological parameter data transmission signals to a second different remotely located monitor such that the first and second transmission frequencies are the same frequency.

20. The measuring system of claim 1 further comprising:
a signal processing unit in said remote R/T unit;
a data storage memory forming a part of said signal processing unit; and
said processing unit containing software enabling comparisons of reference data stored in said memory, relating to medical issues regarding the patient/athlete body parameters, with the patient/athlete parameters being monitored.

21. The measuring system of claim 20 further comprising:
a plurality of physiological/biological parameters being measured by said transducer and being transmitted by said electronic unit; and
an analog switch for controllably switching between said plurality of transmitted plurality of physiological/biological parameters for selecting a particular parameter for monitoring.

22. The measuring system of claim 21 further comprising:
software contained in said processing unit in said remote R/T unit for instructing said analog switch to transmit a desired physiological/biological parameter.

23. The measuring system of claim 20 further comprising:
a plurality of physiological/biological parameters being measured by said transducer and being transmitted by said electronic unit;
each of said parameters being transmitted at a different frequency; and
a plurality of frequency band pass filters in said signal processing unit at said remote R/T unit; and
a frequency selector enabling a user of said monitor to select a given frequency to monitor a given transmitted physiological/biological parameter.

24. A method of measuring physiological/biological parameters of an animate body comprising the steps of:
affixing at least one transducer to said animate body for generating a signal representing a physiological/biological parameter of said body;
coupling an electronic unit to each said transducer for receiving said physiological/biological parameter signals from said at least one transducer and wirelessly transmitting said parameter signals at a first frequency;
receiving said wirelessly transmitted parameter signals from said electronic unit with at least one remote R/T unit; and
coupling a display unit to said R/T unit for visually displaying said parameter signals.

25. The method of claim 24 further comprising the steps of:
physically attaching said transducer to said animate body; and
removably attaching said electronic unit to said transducer.

26. The method of claim 24 further comprising the step of forming said transducer and said electronics unit as a single unit to create a transponder as an intelligent sensor.

27. The method of claim 26 further comprising the step of embedding said intelligent sensor under the skin of said animate body.

28. The method of claim 26 further comprising the step of adding an additional signal to said transmitted parameter signals that identifies the transponder transmitting the parameter signals thereby identifying the animate body to which said transponder is attached.

29. The method of claim 24 further comprising the steps of:
operating a hardwired LAN system at said first frequency;
coupling at least two nodes to said LAN system;
locating a first remote R/T unit at one of said nodes for receiving and transmitting RF signals to and from said LAN system at said first frequency;
transmitting said received physiological/biological parameter signals with said transponder as RF signals to said first remote R/T unit at said first frequency; and
receiving said RF signals from said hardwired LAN system with a second R/T unit located at another one of said LAN nodes.

30. The method of claim 28 further comprising the step of utilizing, as said second R/T unit, at least one of the class of RF signal receivers including, but not limited to, personal digital assistants (PDA's), personal computers (PC's), pagers, and tablet PC's.

31. The method of claim 24 further comprising the step of forming at least one of said remote R/T units as a portable unit that can be carried by personnel to monitor in real time said physiological/biological parameters remotely from the source of said parameter signals thereby enabling a rapid response to changing conditions of said animate body.

32. The method of claim 24 further comprising the steps of:
operating said remote R/T unit at said first RF frequency; and
providing a first plug-in unit that enables said remote R/T unit to communicate with said transponder at said first RF frequency.

33. The method of claim 32 further comprising the steps of:
operating an existing LAN system at a second frequency different than said first frequency; and
inserting a second plug-in unit in said first plug-in unit for converting said first frequency to said second LAN frequency thereby enabling said remote RF R/T unit to communicate with said LAN system at said second frequency.

34. The method of claim 33 further comprising the step of associating identification signals with each R/T unit at each node of said LAN network to enable personnel receiving said RF signals from any node to identify the node of the LAN network that is transmitting the RF signal thereby enabling said personnel to identify the location of the transponder transmitting the physiological/biological parameter signals.

35. A method of measuring physiological/biological parameters of an animate body comprising the steps of:
affixing a plurality of transponders to an animate body such as a patient/athlete;
including in each transponder (1) a transducer for sensing a physiological/biological parameter of said body and generating an electronic signal representing said sensed parameter and (2) an electronic unit for receiving said physiological/biological parameter signal and wirelessly transmitting said parameter signal at a first frequency;
associating a signal with each wireless transmission to identify each said transponder;
locating a receiver/transmitter unit on said animate body for receiving each wireless transmission from each of said transponders and retransmitting each of said received wireless transmissions to at least one remote RF signal receiver at a given RF frequency; and
coupling a display unit to said remote RF signal receiver for visually displaying each of said physiological/biological parameter signals.

36. The method of claim 24 further including the steps of:
generating video images of said animate body; and
transmitting said video images in real time to a remote location for viewing in conjunction with said transmitted physiological/biological parameters of said animate body.

37. The method of claim 36 further comprising the step of transmitting said video images at a frequency different from said first transmission frequency of said physiological/biological parameters of said animate body.

38. The method of claim 36 further comprising the step of transmitting said video images at the same frequency as said first transmission frequency of said physiological/biological parameters of said animate body.

39. The method of claim 37 further comprising the step of separating said different frequencies for viewing with filter means located at said remotely located monitors.

40. The method of claim 37 further comprising the steps of:
receiving and displaying the patient physiological/biological parameters with a first monitor; and
receiving and displaying the patient video images with a second monitor separate from said first monitor.

41. The method of claim 37 further comprising the step of displaying both said patient video images and said patient physiological/biological parameters on a single remotely located monitor.

42. The method of claim 38 further comprising the steps of:
connecting said video image transmission signals directly to a first remotely located monitor along a first transmission path for display; and
connecting said patient physiological/biological parameter data transmission signals to a second different remotely located monitor along a second different transmission path such that the first and second transmission frequencies are the same frequency.
